SEQUENCE LISTING (11 sequences)

SEQ ID NO: 1 - DNA, 1617 bp, Sorghum bicolor
  source 1..1617: mol_type = genomic DNA
  variation 122
atggcgacctactactcttcgcggcgtccatgcaacgcctgcagcacgaaggcgatggccgggagcgtggtcggcgagcccgtcgtgctggggcagagggtgacggtgctgacggtggacgacggcggcatccgtggtctcatccccggaaccatccttgccttcctcgaggcccggctgcaggagctggacgggccggaggttaggctcgcggactacttcgactacatcgccgggacgagcaccggcgggctcatcaccgccatgctcaccgcgcccggcaaggacaggcggcctctctacgctgccaaggacatcaaccaattctacatggagaattgccctcgcatcttccctcaaaagtgagtccgatcatcccggccggccgccattgttggcctcgcattattgatcgatcatgcatgcatgcatgcatgtgatgtgtaggagcagcaggcttgcggccgccatgtccgcgctgaggaagccaaggtacaacggcaagtgcctccgtaacctgatcatgagcatgctcggcgagacgagggtgagcgacacgctcaccaacgtcatcatccctaccttcgacgtcaggctgctgcagcccatcatcttctccacctacgacgtacgtacgccgccggccgccgtcgtcatgaataatcaatcagctgtacattattgcacatggctgcatgcctactgaacatgtttaatttgcaggccaagagcatgcctctgaagaacgcgctgctctccgacgtgtgcatcggcacgtccgccgcgccgacctacctcccggcgcactacttccagaccaaggacgccggcagtggcaaggaacgcgagtacaacctcatcgacggcggtgtcgccgccaacaatccggtaattaattaactgaaaacgaacgaacgcaattccatatccattgtccagaagatcgatgctaattgttgcttaggcacgtcgttgccgccggccgattgcattgcacttgcacatgcgtagacgatggttgcgatgacgcagatcaccaagaagatgcttgccagcaaggagaaggccgaggagctgtacccagtgaagccgtggaactgccgcaagttcctggtgctgtccatcgggacggggtcgacgtcggagcagggcctgtacacggcgcggcagtgctcgcggtggggcatctgccggtggatccggaacaacggcatggcccccatcatcgacatcttcatggcggcgagctcggacctggtggacatccacgtcgccgcgatgttccagtcgctccacagcgacggcgactacctgcgcatccaggacaactcgctgcacggcgccgcggccaccgtggacgcggcgacgccggagaacatgcggacgctcgtcgggatcggggagcggatgctggcgcagcgggtgtccagggtcaacgtggagacagggaggtacgaaccggtgcctggggaaggaagcaacgctgatgcgctcgctgggatcgcaaggcagctctcggaggagaggaggacaaggctcgcgcgccgcacctccgccatcgtcagctccggtggtgcctctagacgtacgtgtgcctcaaaggtctccaatgtctaa

SEQ ID NO: 2 - protein, 437 aa, Sorghum bicolor
  VARIANT 41
MATYYSSRRPCNACSTKAMAGSVVGEPVVLGQRVTVLTVDDGGIRGLIPGTILAFLEARLQELDGPEVRLADYFDYIAGTSTGGLITAMLTAPGKDRRPLYAAKDINQFYMENCPRIFPQKSSRLAAAMSALRKPRYNGKCLRNLIMSMLGETRVSDTLTNVIIPTFDVRLLQPIIFSTYDAKSMPLKNALLSDVCIGTSAAPTYLPAHYFQTKDAGSGKEREYNLIDGGVAANNPTMVAMTQITKKMLASKEKAEELYPVKPWNCRKFLVLSIGTGSTSEQGLYTARQCSRWGICRWIRNNGMAPIIDIFMAASSDLVDIHVAAMFQSLHSDGDYLRIQDNSLHGAAATVDAATPENMRTLVGIGERMLAQRVSRVNVETGRYEPVPGEGSNADALAGIARQLSEERRTRLARRTSAIVSSGGASRRTCASKVSNV

SEQ ID NO: 3 - DNA, 216 bp, Sorghum bicolor
  source 1..216: mol_type = genomic DNA
  variation 107: allele = a or g
tcttcgcggcgtccatgcaacgcctgcagcacgaaggcgatggccgggagcgtggtcggcgagcccgtcgtgctggggcagagggtgacggtgctgacggtggacgncggcggcatccgtggtctcatccccggaaccatccttgccttcctcgaggcccggctgcaggagctggacgggccggaggttaggctcgcggactacttcgactacatc

SEQ ID NO: 4 - DNA, 40 bp, Synthetic construct
  source 1..40: mol_type = other DNA; note = primer
gaaggtgaccaagttcatgctcggtgctgacggtggacga

SEQ ID NO: 5 - DNA, 39 bp, Synthetic construct
  source 1..39: mol_type = other DNA; note = primer
gaaggtcggagtcaacggattggtgctgacggtggacgg

SEQ ID NO: 6 - DNA, 22 bp, Synthetic construct
  source 1..22: mol_type = other DNA; note = primer
aggcaaggatggttccggggat

SEQ ID NO: 7 - DNA, 1314 bp, Sorghum bicolor
  source 1..1314: mol_type = other DNA
  variation 122: allele = A
atggcgacctactactcttcgcggcgtccatgcaacgcctgcagcacgaaggcgatggccgggagcgtggtcggcgagcccgtcgtgctggggcagagggtgacggtgctgacggtggacgacggcggcatccgtggtctcatccccggaaccatccttgccttcctcgaggcccggctgcaggagctggacgggccggaggttaggctcgcggactacttcgactacatcgccgggacgagcaccggcgggctcatcaccgccatgctcaccgcgcccggcaaggacaggcggcctctctacgctgccaaggacatcaaccaattctacatggagaattgccctcgcatcttccctcaaaagagcagcaggcttgcggccgccatgtccgcgctgaggaagccaaggtacaacggcaagtgcctccgtaacctgatcatgagcatgctcggcgagacgagggtgagcgacacgctcaccaacgtcatcatccctaccttcgacgtcaggctgctgcagcccatcatcttctccacctacgacgccaagagcatgcctctgaagaacgcgctgctctccgacgtgtgcatcggcacgtccgccgcgccgacctacctcccggcgcactacttccagaccaaggacgccggcagtggcaaggaacgcgagtacaacctcatcgacggcggtgtcgccgccaacaatccgacgatggttgcgatgacgcagatcaccaagaagatgcttgccagcaaggagaaggccgaggagctgtacccagtgaagccgtggaactgccgcaagttcctggtgctgtccatcgggacggggtcgacgtcggagcagggcctgtacacggcgcggcagtgctcgcggtggggcatctgccggtggatccggaacaacggcatggcccccatcatcgacatcttcatggcggcgagctcggacctggtggacatccacgtcgccgcgatgttccagtcgctccacagcgacggcgactacctgcgcatccaggacaactcgctgcacggcgccgcggccaccgtggacgcggcgacgccggagaacatgcggacgctcgtcgggatcggggagcggatgctggcgcagcgggtgtccagggtcaacgtggagacagggaggtacgaaccggtgcctggggaaggaagcaacgctgatgcgctcgctgggatcgcaaggcagctctcggaggagaggaggacaaggctcgcgcgccgcacctccgccatcgtcagctccggtggtgcctctagacgtacgtgtgcctcaaaggtctccaatgtctaa

SEQ ID NO: 8 - DNA, 1616 bp, Sorghum bicolor
  source 1..1616: mol_type = genomic DNA
atggcgacctactactcttcgcggcgtccatgcaacgcctgcagcacgaaggcgatggccgggagcgtggtcggcgagcccgtcgtgctggggcagagggtgacggtgctgacggtggacggcggcggcatccgtggtctcatccccgaaccatccttgccttcctcgaggcccggctgcaggagctggacgggccggaggttaggctcgcggactacttcgactacatcgccgggacgagcaccggcgggctcatcaccgccatgctcaccgcgcccggcaaggacaggcggcctctctacgctgccaaggacatcaaccaattctacatggagaattgccctcgcatcttccctcaaaagtgagtccgatcatcccggccggccgccattgttggcctcgcattattgatcgatcatgcatgcatgcatgcatgtgatgtgtaggagcagcaggcttgcggccgccatgtccgcgctgaggaagccaaggtacaacggcaagtgcctccgtaacctgatcatgagcatgctcggcgagacgagggtgagcgacacgctcaccaacgtcatcatccctaccttcgacgtcaggctgctgcagcccatcatcttctccacctacgacgtacgtacgccgccggccgccgtcgtcatgaataatcaatcagctgtacattattgcacatggctgcatgcctactgaacatgtttaatttgcaggccaagagcatgcctctgaagaacgcgctgctctccgacgtgtgcatcggcacgtccgccgcgccgacctacctcccggcgcactacttccagaccaaggacgccggcagtggcaaggaacgcgagtacaacctcatcgacggcggtgtcgccgccaacaatccggtaattaattaactgaaaacgaacgaacgcaattccatatccattgtccagaagatcgatgctaattgttgcttaggcacgtcgttgccgccggccgattgcattgcacttgcacatgcgtagacgatggttgcgatgacgcagatcaccaagaagatgcttgccagcaaggagaaggccgaggagctgtacccagtgaagccgtggaactgccgcaagttcctggtgctgtccatcgggacggggtcgacgtcggagcagggcctgtacacggcgcggcagtgctcgcggtggggcatctgccggtggatccggaacaacggcatggcccccatcatcgacatcttcatggcggcgagctcggacctggtggacatccacgtcgccgcgatgttccagtcgctccacagcgacggcgactacctgcgcatccaggacaactcgctgcacggcgccgcggccaccgtggacgcggcgacgccggagaacatgcggacgctcgtcgggatcggggagcggatgctggcgcagcgggtgtccagggtcaacgtggagacagggaggtacgaaccggtgcctggggaaggaagcaacgctgatgcgctcgctgggatcgcaaggcagctctcggaggagaggaggacaaggctcgcgcgccgcacctccgccatcgtcagctccggtggtgcctctagacgtacgtgtgcctcaaaggtctccaatgtctaa

SEQ ID NO: 9 - DNA, 1314 bp, Sorghum bicolor
  source 1..1314: mol_type = other DNA
atggcgacctactactcttcgcggcgtccatgcaacgcctgcagcacgaaggcgatggccgggagcgtggtcggcgagcccgtcgtgctggggcagagggtgacggtgctgacggtggacggcggcggcatccgtggtctcatccccggaaccatccttgccttcctcgaggcccggctgcaggagctggacgggccggaggttaggctcgcggactacttcgactacatcgccgggacgagcaccggcgggctcatcaccgccatgctcaccgcgcccggcaaggacaggcggcctctctacgctgccaaggacatcaaccaattctacatggagaattgccctcgcatcttccctcaaaagagcagcaggcttgcggccgccatgtccgcgctgaggaagccaaggtacaacggcaagtgcctccgtaacctgatcatgagcatgctcggcgagacgagggtgagcgacacgctcaccaacgtcatcatccctaccttcgacgtcaggctgctgcagcccatcatcttctccacctacgacgccaagagcatgcctctgaagaacgcgctgctctccgacgtgtgcatcggcacgtccgccgcgccgacctacctcccggcgcactacttccagaccaaggacgccggcagtggcaaggaacgcgagtacaacctcatcgacggcggtgtcgccgccaacaatccgacgatggttgcgatgacgcagatcaccaagaagatgcttgccagcaaggagaaggccgaggagctgtacccagtgaagccgtggaactgccgcaagttcctggtgctgtccatcgggacggggtcgacgtcggagcagggcctgtacacggcgcggcagtgctcgcggtggggcatctgccggtggatccggaacaacggcatggcccccatcatcgacatcttcatggcggcgagctcggacctggtggacatccacgtcgccgcgatgttccagtcgctccacagcgacggcgactacctgcgcatccaggacaactcgctgcacggcgccgcggccaccgtggacgcggcgacgccggagaacatgcggacgctcgtcgggatcggggagcggatgctggcgcagcgggtgtccagggtcaacgtggagacagggaggtacgaaccggtgcctggggaaggaagcaacgctgatgcgctcgctgggatcgcaaggcagctctcggaggagaggaggacaaggctcgcgcgccgcacctccgccatcgtcagctccggtggtgcctctagacgtacgtgtgcctcaaaggtctccaatgtctaa

SEQ ID NO: 10 - protein, 437 aa, Sorghum bicolor
MATYYSSRRPCNACSTKAMAGSVVGEPVVLGQRVTVLTVDGGGIRGLIPGTILAFLEARLQELDGPEVRLADYFDYIAGTSTGGLITAMLTAPGKDRRPLYAAKDINQFYMENCPRIFPQKSSRLAAAMSALRKPRYNGKCLRNLIMSMLGETRVSDTLTNVIIPTFDVRLLQPIIFSTYDAKSMPLKNALLSDVCIGTSAAPTYLPAHYFQTKDAGSGKEREYNLIDGGVAANNPTMVAMTQITKKMLASKEKAEELYPVKPWNCRKFLVLSIGTGSTSEQGLYTARQCSRWGICRWIRNNGMAPIIDIFMAASSDLVDIHVAAMFQSLHSDGDYLRIQDNSLHGAAATVDAATPENMRTLVGIGERMLAQRVSRVNVETGRYEPVPGEGSNADALAGIARQLSEERRTRLARRTSAIVSSGGASRRTCASKVSNV

SEQ ID NO: 11 - DNA, 1314 bp, Sorghum bicolor
  source 1..1314: mol_type = other DNA
  variation 122: allele = a
  variation 123: allele = t
atggcgacctactactcttcgcggcgtccatgcaacgcctgcagcacgaaggcgatggccgggagcgtggtcggcgagcccgtcgtgctggggcagagggtgacggtgctgacggtggacgatggcggcatccgtggtctcatccccggaaccatccttgccttcctcgaggcccggctgcaggagctggacgggccggaggttaggctcgcggactacttcgactacatcgccgggacgagcaccggcgggctcatcaccgccatgctcaccgcgcccggcaaggacaggcggcctctctacgctgccaaggacatcaaccaattctacatggagaattgccctcgcatcttccctcaaaagagcagcaggcttgcggccgccatgtccgcgctgaggaagccaaggtacaacggcaagtgcctccgtaacctgatcatgagcatgctcggcgagacgagggtgagcgacacgctcaccaacgtcatcatccctaccttcgacgtcaggctgctgcagcccatcatcttctccacctacgacgccaagagcatgcctctgaagaacgcgctgctctccgacgtgtgcatcggcacgtccgccgcgccgacctacctcccggcgcactacttccagaccaaggacgccggcagtggcaaggaacgcgagtacaacctcatcgacggcggtgtcgccgccaacaatccgacgatggttgcgatgacgcagatcaccaagaagatgcttgccagcaaggagaaggccgaggagctgtacccagtgaagccgtggaactgccgcaagttcctggtgctgtccatcgggacggggtcgacgtcggagcagggcctgtacacggcgcggcagtgctcgcggtggggcatctgccggtggatccggaacaacggcatggcccccatcatcgacatcttcatggcggcgagctcggacctggtggacatccacgtcgccgcgatgttccagtcgctccacagcgacggcgactacctgcgcatccaggacaactcgctgcacggcgccgcggccaccgtggacgcggcgacgccggagaacatgcggacgctcgtcgggatcggggagcggatgctggcgcagcgggtgtccagggtcaacgtggagacagggaggtacgaaccggtgcctggggaaggaagcaacgctgatgcgctcgctgggatcgcaaggcagctctcggaggagaggaggacaaggctcgcgcgccgcacctccgccatcgtcagctccggtggtgcctctagacgtacgtgtgcctcaaaggtctccaatgtctaa

(19) United States
(12) Patent Application Publication
(10) Pub. No.: US 2026/0139265 A1
(43) Pub. Date: May 21, 2026

(54) HAPLOID INDUCING SORGHUM PLANT

(71) Applicant: Advanta Holdings B.V., Breda (NL)

(72) Inventors: Pedro Alejandro Pardo, Salta (AR); Maria de la Paz Arrieta Montiel, College Station, TX (US); Hernan Gabriel Bondino, College Station, TX (US)

(21) Appl. No.: 19/119,565

(22) PCT Filed: Oct. 18, 2023
(86) PCT No.: PCT/EP2023/078949

§ 371 Date: Apr. 9, 2025

Related U.S. Application Data
(60) Provisional application No. 63/417,078, filed on Oct. 18, 2022.

(51) Int. Cl. C12N 15/82; C12N 9/18; C12Q 1/6876

(52) U.S. Cl. CPC C12N 15/8262; C12N 9/18; C12Q 1/6876; C12Q 2600/156

(57) ABSTRACT

The present invention relates to a polynucleotide encoding a mutated patatin phospholipase protein, which mutated protein leads to an improved haploid induction rate. The invention further relates to the mutant patatin phospholipase protein and to methods and compositions for improved plant breeding using haploid induction. Sorghum plants which are capable of haploid induction with improved induction performance are described.

### CROSS-REFERENCE TO RELATED APPLICATIONS

This application claims priority to U.S. Provisional Application No. 63/417,078, filed Oct. 18, 2022 which is incorporated herein by reference in its entirety.

### INCORPORATION OF SEQUENCE LISTING

A sequence listing contained in the file name “Methods and Compositions of Haploid Plants_WO_ST26.txt” which is 20,480 bytes and was created on Sep. 29, 2023, is filed electronically herewith and incorporated by reference in its entirety.

### FIELD OF THE INVENTION

The present invention relates to the field of simplified breeding in plants by means of molecular techniques. In particular, the present invention relates to a simplified breeding technique in plants by introducing haploidy.

### BACKGROUND

Developing new cultivars with traditional breeding methods is slow since it can take several generations of inbreeding. Besides time, selection efficiency in early generations is affected due to heterozygosity. Plant breeders can use haploid induction lines and double haploids to speed the inbreeding process and increase the selection efficiency among other processes in breeding. A double haploid (DH) is a genotype formed when a cell undergoes chromosome doubling. Availability of double haploids increases the efficiency in plant breeding. Thus, artificial production of double haploids is important in plant breeding. Generally, a conventional breeding cycle takes at least five to six generations to achieve approximately complete homozygosity for the trait of interest, whereas in the case of double haploids this time period is reduced to one generation only.

Sorghum is the fifth most important crop after corn, rice, wheat and pearl millet in the world. Conventionally, sorghum breeding relies on multiple generations of self-pollination to achieve the adequate levels of homozygosity for hybrid evaluation which adds several years and great cost to the breeding process. Thus, Double Haploidy is the key technology to speed up the breeding process in the sorghum breeding program. Two double haploid lines (SMHI01 and SMHI02) that are able to generate haploids at frequency of 1-2% are known (Discovery of Sorghum Haploid Induction System, Tanveer et al; Methods Mol Biol; 2019; 1931:49-59. doi: 0.1007/978-1-4939-9039-9_4).

Patent application US 2019/0390213 A1 by KWS Saat also tries to solve the challenge of long breeding process in sorghum by inducing double haploidy in sorghum by introducing mutations in its genome. It mentions that the exchange of the amino acid serine for leucine at amino acid position 291 according to the amino acid sequence of the patatin phospholipase protein in sorghum plants results in a haploid induction rate of more than 1.5%. It further specifically mentions that an average efficiency obtained is 1.5% when the mutation was homozygous and 1.2% when the mutation was heterozygous.

The frequencies of the above-described methods of inducing haploidy in sorghum are too low and thus there is still a need in the field for methods to obtain haploid plants with greater frequency or efficiency.

### SUMMARY

In the research leading to the present invention, it was found that other modifications in the patatin phospholipase protein lead to a higher efficiency in inducing haploidy.

The present invention thus relates to a novel polynucleotide having a mutation that leads to an increased efficiency in the production of haploid plants. The mutation in the polynucleotide leads to a mutated patatin phospholipase protein, which comprises in particular aspartic acid (D) at position 41 instead of glycine (G). The mutated protein is also part of this invention.

The present invention also relates to novel sorghum plants which are capable of inducing haploidy as a result of the mutated patatin phospholipase protein.

The present invention relates to the simplified breeding process in sorghum plants which are capable of inducing haploidy.

The present invention relates to a mutation in a plant that produces a plant with haploid frequency more than 2%. The present invention specifically provides a sorghum plant and a mutation in sorghum plant that has haploidy efficiency of more than 5%, more preferably 2%-10%. The present invention specifically provides a sorghum plant and mutation in sorghum plant that has haploidy efficiency of 5%.

The present invention relates to a plant characterized in that the mutant patatin phospholipase is encoded by the nucleotide sequence according to SEQ ID NO: 1 or by a nucleotide sequence which is homologous or identical to SEQ ID NO: 1 or is encoded by a nucleotide sequence which hybridizes with the sequence complementary to the nucleotide sequence according to SEQ ID NO: 1 under relevant stringent conditions, wherein the homologous nucleotide sequence comprises the mutation that leads to the increased frequency of haploidy induction.

The present invention relates to a plant characterized in that the patatin phospholipase is encoded by the nucleotide sequence according to SEQ ID NO: 1 or by a nucleotide sequence which is at least 80%-99% identical to SEQ ID NO: 1 and comprises the said mutation. The present invention relates to a plant characterized in that the patatin phospholipase is encoded by the nucleotide sequence according to SEQ ID NO: 1 or by a nucleotide sequence which is at least 90%-99%, in particular 95%-99%, 96%-99%, 97%-99%, 98%-99% identical to SEQ ID NO: 1 and comprises the said mutation.

The present invention relates to a sorghum plant comprising the modified patatin phospholipase with nucleotide sequence as mentioned in Alternate Codons 1 (SEQ ID NO: 11) that codes for an amino acid sequence of SEQ ID NO: 2. The present invention relates to a plant which comprises the modified patatin phospholipase with the amino acid sequence shown in SEQ ID NO: 2 or a homologous amino acid sequence which comprises the amino acid change. The present invention relates to a sorghum plant comprising the amino acid sequence shown in SEQ ID NO: 2 or a homologous amino acid sequence which comprises the amino acid change.

The present invention relates to a modification in patatin phospholipase that is the cause of the suitability as haploid inducer or increases the haploid efficiency of the plant.

The invention also relates to a genetic marker that is indicative of a phenotype associated with the mutation in patatin phospholipase causing haploidy. The genetic marker is capable of identifying the mutation in SEQ ID NO: 1.

A method of producing a plant with haploid induction activity according to the present invention may comprise introducing the mutation of the invention into a plant.

A method of obtaining a plant capable of inducing haploidy or having an increased induction rate of >2% or preferably >5%, more preferably 2%-10% comprises the following steps: (a) mutagenizing a sorghum cell or introducing a nucleic acid corresponding to the mutation in SEQ ID NO: 1/Alternate Codons and/or coding for a polypeptide of SEQ ID NO: 2 into the cell and then regenerating the plants from the mutagenized or transformed plant cells or mutagenizing plants; (b) identifying the plant having the mutation of SEQ ID NO: 1 or Alternate Codons or encodes for a polypeptide of SEQ ID NO: 2.

The present invention further provides a method of detecting an allele or gene or SEQ ID NO: 1 comprising the mutation according to the present invention in a plant. Diagnostic detection methods of the present invention may, for example, include polymerase chain reaction (PCR) amplification of specific regions of the genome of the plant, Sanger sequencing, or hybridization methods.

The present invention further provides a method of selecting a plant comprising the novel mutation of the invention. Methods of selection of the present invention may, for example, include flow cytometry, phenotypic selection, molecular methods or any other relevant applicable method.

The present invention provides a method of obtaining a haploid plant comprising the following steps: (a) crossing a plant comprising a polypeptide according to the amino acid sequence of SEQ ID NO: 2 with another plant that may or may not comprise the mutated polypeptide of SEQ ID NO: 2; (b) selecting a fertilized haploid seed or embryo; (c) producing a haploid plant from the seed or embryo selected in step (b).

The present invention further provides the use of the nucleic acid according to SEQ ID NO: 1 for detecting the mutation in the gene. Alternatively, any other nucleic acid molecules with 99% sequence identity to SEQ ID NO: 1 and coding for the mutated patatin phospholipase protein of SEQ ID NO: 2 can be used. In a further embodiment, a marker allele which is coupled to the mutation is used as a molecular marker for detecting the mutation in the gene.

The present invention also provides use of the plant comprising the mutated polypeptide of SEQ ID NO: 2 for the production of a haploid fertilized seed or embryo or a haploid plant. The present invention also provides use of the plant comprising the mutated polynucleotide of SEQ ID NO: 1 or Alternate Codons for the production of a haploid fertilized seed or embryo or a haploid plant.

The present invention provides a sorghum plant (Sorghum bicolor (L.) Moench SV2081) comprising a mutated patatin phospholipase as compared to wild type and has haploidy induction activity and a representative sample of seeds of which is deposited under the Budapest Treaty with NCIMB Ltd, Ferguson Building, Craibstone Estate, Bucksburn, Aberdeen AB21 9YA, UK on 23 September 2022 under deposit accession number NCIMB 44032.

### BRIEF DESCRIPTION OF THE DRAWINGS

FIG. 1 illustrates flow cytometry experiments that identify diploid plants (A) and haploid plants (B).

### DETAILED DESCRIPTION

As used herein, “plant” refers to a whole plant or a cell or tissue culture derived from a plant, comprising any of: whole plants, plant components or organs (e.g., leaves, stems, roots, etc.), plant tissues, seeds, plant cells, and/or progeny of the same. A progeny plant can be from any filial generation, e.g., F1, F2, F3, F4, F5, F6, F7, etc. A plant cell is a biological cell of a plant, taken from a plant or derived through culture from a cell taken from a plant. Plant parts include harvestable parts and parts useful for propagation of progeny plants. Plant parts useful for propagation include, for example and without limitation: seed; fruit; a cutting; a seedling; a tuber; and a rootstock. A harvestable part of a plant may be any useful part of a plant, including, for example and without limitation: flower; pollen; seedling; tuber; leaf; stem; fruit; seed; and root. Plant cells, as used herein, includes protoplasts and protoplasts with a cell wall. A plant cell may be a protoplast, a gamete producing cell, or a cell or collection of cells that can regenerate into a whole plant.

An “endogenous” gene or protein sequence refers to a non-recombinant sequence of an organism as the sequence occurs in the organism before human-induced mutation of the sequence. A “mutated” sequence refers to a human-altered sequence. Examples of human-induced mutation include exposure of an organism to a high dose of chemical, radiological, or insertional mutagen for the purposes of selecting mutants, as well as recombinant alteration of a sequence. Examples of human-induced recombinant alterations can include, e.g., fusions, insertions, deletions, and/or changes to the sequence.

As used herein, “transgenic” refers to a plant cell, a plant, a plant part, or a seed whose genome has been altered by the stable integration of exogenous DNA. A transgenic line includes a plant regenerated from an originally-transformed plant cell and progeny transgenic plants from later generations or crosses of a transformed plant. As used herein, a “transgene” refers to a polynucleotide that has been transferred into a genome by any method known in the art. In one aspect, a transgene is an exogenous polynucleotide. In one aspect, a transgene is an endogenous polynucleotide that is integrated into a new genomic locus where it is not normally found.

The term “promoter” refers to regions or sequence located upstream and/or downstream from the start of transcription and which are involved in recognition and binding of RNA polymerase and other proteins to initiate transcription. A “plant promoter” is a promoter capable of initiating transcription in plant cells. A plant promoter can be, but does not have to be, a nucleic acid sequence originally isolated from a plant.

As used herein, a “haploid” cell or nucleus comprises a single set of unpaired chromosomes (x). In contrast, a “diploid” cell or nucleus comprises two complete sets of chromosomes (2x) that are capable of homologous pairing. The haploid number of chromosomes can be represented by “n,” and the diploid number of chromosomes can be represented by “2n.” For example, in a diploid species such as corn, n=x=10, and 2n=2x=20. A polyploid cell or nucleus comprises more than two complete sets of chromosomes. For example, some wheat lines are hexaploids, meaning they contain three sets of paired chromosomes (2n=6x=42). Both diploid and polyploid cells and nuclei can be reduced to haploid states. A “haploid induction rate (HIR)” means the number of haploid seeds divided by the total number of seeds tested from a cross-pollination with a haploid inducer. Comparing the HIR of different inducers is a common practice in the art (See Trentin et al. “Breeding Maize Maternal Haploid Inducers.” Plants (Basel). 2020 May 12; 9 (5): 614.

The phrase “nucleic acid” or “polynucleotide sequence” refers to a single or double-stranded polymer of deoxyribonucleotide or ribonucleotide bases read from the 5′ to the 3′ end. Nucleic acids may also include modified nucleotides that permit correct read through by a polymerase, and/or formation of double-stranded duplexes, and do not significantly alter expression of a polypeptide encoded by that nucleic acid.

The phrase “nucleic acid sequence encoding” refers to a nucleic acid which directs the expression of a specific protein or peptide. The nucleic acid sequences include both the DNA strand sequence that is transcribed into RNA and the RNA sequence that is translated into protein. The nucleic acid sequences include both the full length nucleic acid sequences as well as non-full length sequences derived from the full length sequences. It should be further understood that the sequence includes the degenerate codons of the native sequence or sequences which may be introduced to provide codon preference in a specific host cell.

Two nucleic acid sequences or polypeptides are said to be “identical” if the sequence of nucleotides or amino acid residues, respectively, in the two sequences is the same when aligned for maximum correspondence as described below. The term “complementary to” is used herein to mean that the sequence is complementary to all or a portion of a reference polynucleotide sequence.

Examples of algorithms that are suitable for determining percent sequence identity and sequence similarity are the BLAST and BLAST 2.0 algorithms, which are described in Altschul et al., Nuc. Acids Res. 25:3389-3402 (1977), and Altschul et al., J. Mol. Biol. 215:403-410 (1990), respectively. Software for performing BLAST analyses is publicly available on the Web through the National Center for Biotechnology Information (at ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, (1989)) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has at least 25% sequence identity to a designated reference sequence. Alternatively, percent identity can be any integer from 25% to 100%, for example, at least: 25%, 30%, 35%, 40%, 45%, 50%, 55%, 60%, 65%, 70%, 75%, 80%, 85%, 90%, 95%, or 99% compared to a reference sequence using the programs described herein; preferably BLAST using standard parameters, as described below. One of skill will recognize that the percent identity values above can be appropriately adjusted to determine corresponding identity of proteins encoded by two nucleotide sequences by taking into account codon degeneracy, amino acid similarity, reading frame positioning and the like. The term “alternate codon” means any or all nucleotide sequences that can yield the amino acid sequence 95% similar, more preferably 99% to the SEQ ID NO: 2 provided the amino acid sequence has maintained the mutation. Alternate codons include but not limited to SEQ ID NO: 11.

Haploid plants often form aberrant floral structures and are unable to proceed through meiosis due to the absence of one set of homologous chromosomes. It is often desirable to convert a haploid plant to a diploid plant (a “doubled haploid”) in a process known as “haploid doubling” or “chromosome doubling.” Haploid doubling allows the generation of a plant that is homozygous at all loci in the nuclear genome in a single generation, eliminating the numerous cycles of inbreeding necessary by conventional methods to achieve practical levels of homozygosity. In one aspect, a haploid plant provided herein is converted to a doubled haploid plant. In one aspect, a method of chromosome doubling provided herein comprises the use of a chromosome doubling agent selected from the group consisting of nitrous oxide (N2O) gas, colchicine, oryzalin, amiprophosmethyl, trifluralin, caffeine, and pronamide. See for example, Doubled Haploid Production in Crop Plants: A Manual (Eds. M. Maluszynski, K. J. Kasha, B. P. Forster, and I. Szarejko (2003), Kluwer Academic Publishers); Prigge and Melchinger, 2012, Plant Cell Culture Protocols, 877:161-172; and Kato and Geiger, 2002, Plant Breeding, 121:370-377 (each of which are incorporated by reference herein in their entireties). In another aspect, a method of chromosome doubling provided herein comprises the use of colchicine. In yet another aspect, a method of chromosome doubling provided herein comprises the use of N20 gas. In still another aspect, a method of chromosome doubling provided herein comprises the use of colchicine or nitrous oxide gas. As used herein, when referring to chromosome count, “doubling” refers to increasing the chromosome number by a factor of two. Confirmation of chromosome doubling can be carried out by FISH or other molecular biology techniques known in the art.

The present invention provides a plant which is capable of inducing haploidy. More specifically the present invention provides a mutant plant of the genus sorghum, hereinafter referred to as sorghum plant which is capable of inducing haploidy. A sorghum haploid inducer line is a sorghum plant that when crossed to a line of the same species generates haploid offspring.

The sorghum plant of the present invention is capable of producing plants, fertilized seeds or embryos having a haploid chromosome set from the cross of the plant of the same genus preferably of the same species which does not possess the property of the haploid inducer. The inventors of the present invention have surprisingly found that the mutated polypeptide with SEQ ID NO: 2 is capable of inducing haploidy or having an increased induction rate in sorghum plant of >2% or preferably >5%, more preferably 2%-10%, thus providing an efficient system for sorghum breeding.

The sorghum plants according to the present invention have modifications which relate to the patatin phospholipase, which either confers the haploid induction property or also improves a naturally present ability for haploid induction or increases the induction performance. The modification in patatin phospholipase causes the suitability of the sorghum plant as the haploid inducer in breeding programs.

Sorghum plants of the present invention have an increased haploid induction rate of >2% or preferably >5%, more preferably 2%-10%.

In a preferred embodiment, the mutated patatin phospholipase is the polypeptide of SEQ ID NO: 2, which is encoded by the polynucleotide of SEQ ID NO: 1/Alternate Codons or by any polynucleotide which is at least 80%-99%, preferably 90%-99%, more preferably 95-99% and most preferably 99% identical to Seq ID NO: 1/Alternate Codons and comprises the mutation.

In another preferred embodiment, the mutated patatin phospoholipase protein of the present invention may be encoded by a homolog patatin phospholipase gene. The present invention provides a sorghum plant with a mutated patatin phospholipase protein of SEQ ID NO: 2 and has the haploid induction rate of >2% or preferably >5%, more preferably 2%-10%. The mutated patatin phospholipase protein of the present invention consists of aspartic acid (D) at the position 41 instead of glycine (G).

In one of the preferred embodiments, the present invention provides the plant that has a mutated patatin phospholipase gene or nucleic acid molecule that encodes a mutated patatin phospholipase protein that consists of aspartic acid at the position 41 instead of glycine. The nucleic acid molecule according to the present invention is characterized in that its presence in a plant results in the plant that can induce haploidy or can increase or improve a haploid induction rate. Preferably, the presence of the nucleic acid molecule according to the present invention in the absence of wild-type protein in the plant results in the plant being able to induce haploidy or improve or increase the haploid induction rate of the plant.

The nucleic acid molecule according to the present invention can be used as the transgene to impart the property of haploid inducer in a plant or to increase the haploid induction in the plant. The nucleic acid according to the present invention may be double stranded or single stranded or circular or linear. This can be synthetic DNA, genomic DNA, cDNA, RNA or mRNA, siRNA, miRNA, wherein nucleobase Uracil occurs in RNA rather than DNA.

Nucleic acid hybridization techniques like DNA hybridization can be used to identify the plants according to the invention to detect the mutation in the patatin phospholipase gene that encodes the polypeptide of SEQ ID No. 2. To achieve such hybridizations such probes should be specific and have length in the range of at least 15-150 nucleotides, preferably in the range of 15-100, 15-95, 15-70, 15-60, 15-50, 15-40, 15-30, 15-25, 15-20 nucleotides. The nucleic acid molecule according to the present invention for the hybridization may also have at least 15, 16, 17, 18, 19 or 20, preferably at least 21, 22, 23, 24 or 25, more preferably at least 30, 35, 40, 45 or 50, and most preferably at least 100, 200, 300, 500 or 1000 nucleotides in length. A detailed guidance on the hybridization of nucleic acids can be found in Tijssen, Laboratory Techniques in Biochemistry and Molecular Biology-Hybridization with Nucleic Acid Probes, Part 1, Chapter 2, “Overview of principles of hybridization and the strategy of nucleic acid assay assays”, Elsevier, New York (1993); and in Current Protocols in Molecular Biology, Chapter 2, Ausubel, et al., eds, Greene Publishing and Wiley Interscience, New York (1995).

The present invention provides the DNA sequence SEQ ID NO: 3 that can be used to design the molecular marker to detect the mutation in SEQ ID NO: 1 or Alternate Codons, wherein A is the preferred allele for the haploid inducer. More preferably the present invention provides primers described in Table 1 to detect the mutation of SEQ ID NO: 1.

TABLE 1
Molecular Primers for a KASP assay used to detect
the mutation of the present invention.

| SEQ | | |
|---|---|---|
| ID | | DESCRIP- |
| NO: | SEQUENCE | TION |
| 3 | TCTTCGCGGCGTCCATGCAACGCCTGCAGCA | Amplicon |
| | CGAAGGCGATGGCCGGGAGCGTGGTCGGCG | |
| | AGCCCGTCGTGCTGGGGCAGAGGGTGACGG | |
| | TGCTGACGGTGGACG[A/G]CGGCGGCATCCG | |
| | TGGTCTCATCCCCGGAACCATCCTTGCCTTCC | |
| | TCGAGGCCCGGCTGCAGGAGCTGGACGGGC | |
| | CGGAGGTTAGGCTCGCGGACTACTTCGAC | |
| | TACATC | |
| 4 | GAAGGTGACCAAGTTCATGCTCGGTGCTGAC | Primer 1 |
| | GGTGGACGA | |
| 5 | GAAGGTCGGAGTCAACGGATTGGTGCTGAC | Primer 2 |
| | GGTGGACGG | |
| 6 | AGGCAAGGATGGTTCCGGGGAT | Reverse |
| | | Primer |

The above-mentioned primers can also be used to amplify the range of modified nucleic acid molecule to identify the mutation according to the present invention using techniques like polymerase chain reaction (PCR), such as Taqman Assay, Allele-Specific PCR with a Blocking reagent (ASB-PCR), PCR-based cleaved amplified polymorphic sequences (CAPS) markers, or Kompetitive allele specific PCR (KASP) assay.

Another aspect of the present invention is to provide vectors that comprises the nucleic acid molecule encoding the mutated patatin phospholipase polypeptide with SEQ ID NO: 2. A vector according to the invention can comprise a mutated patatin phospholipase gene operatively linked to a heterologous promoter or a natural promoter. The vector can be a plasmid, cosmid, phage or any other agent that can be used to transfer the desired nucleic acid molecule into the desired host.

Preferably, the nucleic acid molecule according to the invention is operably linked in an expression vector having one or more regulatory sequences which permit transcription and optionally expression in a prokaryotic or eukaryotic host cell; see, for example, Sambrook et al., Molecular Cloning: A Laboratory Manual 3rd Ed. Cold Spring Harbor Laboratory Press, Cold Spring Harbor, N.Y., 2001.

The methods for the preparation of a desired vector and method of introducing such vectors into the desired host are familiar to a person skilled in the art. There are many methods to introduce the vectors in the desired host this includes but are not limited to transformation, Agrobacterium mediated transformation, transfection, conjugation and like. Preferably, the present invention also provides stable and viable transgenic plants or plants cells or plant parts that have the modified patatin phospholipase gene according to the present invention that codes for the modified patatin phospholipase protein with SEQ ID NO: 2. The present invention also provides the constructs, vectors for the production of such transgenic plants.

The present invention provides a method of producing a haploid plant of the invention. A method of producing a haploid plant of the invention may comprise introducing the mutation of the invention into a plant. Method of obtaining a plant capable of inducing haploidy or having an increased induction rate of >2% or preferably >5%, more preferably 2%-10% comprises the following steps: a) mutagenizing a plant cell or introducing the nucleic acid corresponding to the mutation in SEQ ID NO: 1/Alternate Codons and/or coding for polypeptide of SEQ ID NO: 2 into a plant cell and then regenerating plants from the mutagenized or transformed plant cells, or mutagenizing plants; b) identifying the plant having the mutation of Seq ID NO: 1/Alternate Codons or codes for a polypeptide of SEQ ID NO: 2.

The process of mutagenizing according to the present invention may include conventional mutagenesis methods for example TILLING by UV irradiation, or use of chemicals agents like EMS. The mutagenesis may also be aided by transposons or site directed mutagenesis or any other methods that are available or known to a person skilled in the art.

The present invention also provides the method to detect the plant or polynucleotide comprising the mutation according to the present invention using primer and probes.

The present invention also relates to a plant which can be produced or is produced by the above method. The invention further relates to a part of this plant, wherein a part of a plant can be a fertilized or unfertilized seed, an embryo, a pollen, a tissue, an organ or a plant cell comprising the at least one mutation. The fertilized or unfertilized seed, the embryo or the pollen are suitably produced on the plant. The present invention also includes an offspring of the plant which has the at least one mutation and is suitable for use as a haploid inducer.

According to the present invention, the method of selecting the haploid fertilized seed or embryo can comprise a step of detecting haploidy and the separating of the haploid fertilized seed or embryo from polyploid fertilized seeds or embryos. The detection of haploidy of a fertilized seed or embryo can be phenotypic or genotypic, for example, by providing the inducer with an embryo specific dominant marker which is visible in all diploid offspring but not in the induced haploid offspring. Furthermore, the ploidy status can be determined by flow cytometry. In addition, a completely homozygous pattern of molecular markers indicates haploid plants. The separation can be automated, for example, based on data from the detection of haploidy.

For the purpose of this invention sorghum plant includes any sorghum plant or its part that may or may not have any desired traits including but not limited to drought tolerance, herbicide resistance, increased yield, any disease resistant. The sorghum plant of the present invention may have an additional mutation that may be causative of any additional trait including but not limited to drought tolerance, herbicide resistance or tolerance, disease resistance of tolerance.

The present invention also provides the aspects for the production of double haploid or diploid sorghum plants using the haploid sorghum plants according to the present invention.

### EXAMPLES

The following are non-limiting exemplary embodiments of the present disclosure:

### Example 1. Producing Mutagenized Sorghum Lines with Mutated Patatin Phospholipase

Three batches of sorghum seeds were treated with EMS mutagenic agent. The 5,000 treated seeds were planted in the field, self-pollinated, and seeds were collected from 4,718 plants.

Tilling-by-sequencing (see Spillane et al., “TILLING by Sequencing (TbyS) for targeted genome mutagenesis in crops.” Molecular Breeding 37 (14), 2017.) was used to identify mutations in the patatin phospholipase gene. The G41D mutation was found and further confirmed by Sanger sequencing.

The PCR mixture had a final volume of 15 ul and the following components: 1× reaction buffer (New England Biolabs) 0.2 mM dNTPs (Invitrogen), 0.2 μM of each primer, 0.2 ul Taq DNA polymerase (5 U/μl) (New England Biolabs) and 50 ng of genomic DNA. The PCR reaction was performed in a Veriti 96-Well Fast Thermal Cycler (Applied Biosystems) and amplification conditions were as follows: a step of initial denaturation at 95° C. for 30 seconds followed by 35 cycles at 95° C. for 30 seconds, 60° C. for 20 seconds, and 68° C. for 40 seconds, and a final elongation step at 68° C. for 6 minutes.

Two ul of the DNA product resulting from amplification by PCR was examined by agarose gel electrophoresis to analyze fragment sizes with reference to the molecular weight marker Low DNA Mass Ladder (Invitrogen). The remaining PCR product was cleaned using ExoSAP-IT (Applied Biosystems). Purified DNA was sequenced by the service provider Epoch Life Science, INC using an ABI 3730XL sequencer.

Alternatively, the mutation of the embodiment can also be detected using KASP genotyping techniques with the primers of SEQ ID NOS: 4-6.

### Example 2. Determining Haploidy Efficacy in the Sorghum Plant of the Invention

To determine whether the lines carrying the homozygous mutation of present invention cause haploid induction, a cross was performed. The line carrying the homozygous mutation of the present invention was used as a pollen donor and crossed to a line that is cytoplasmic male sterile and has phenotype A.

The seeds obtained from the cross were planted in a green house. All plants that displayed phenotype A were haploid plants (See Table 2). The experiment was repeated with crossing different inbred background lines to a plant having the mutation of the current invention.

TABLE 2
Screen to determine haploid efficacy

| | | | | | #Candidates | |
|---|---|---|---|---|---|---|
| | | | | | Haploid | |
| | | Total | | | genotypes | |
| | Female in the | seeds | #Phenotyped | Germination | Identified | Induction |
| Year | cross | sowed | Plants | % | (n) | rate |
| 2021 | Proprietary | 1485 | 1040 | 70 | 59 | 5.67% |
| | inbred line 1 | | | | | |
| 2023 | Proprietary | 784 | 570 | 73 | 36 | 6.30% |
| | inbred line 2 | | | | | |
| 2023 | Proprietary | 610 | 440 | 72 | 24 | 5.50% |
| | inbred line 3 | | | | | |

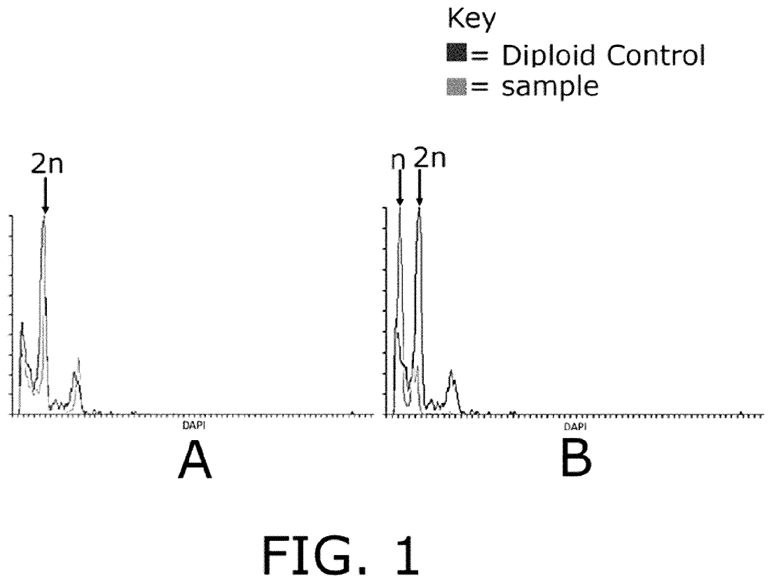

The genotype of the haploid plants were confirmed by flow cytometer analysis of iodide-stained nuclei, in which the fluorescence of each nucleus was quantified (directly proportional to the DNA quantity of the nucleus), showing the haploid plants have half the fluorescence value of the diploid plants. An example of flow cytometer analysis is seen in FIG. 1. A small amount of leaf tissue was harvested from each putative haploid and co-chopped in nuclei isolation buffer, with a sorghum sugarcane hybrid as the standard, for ploidy comparisons. The slurry was filtered through a 30 μm mesh and stained with propidium iodide. The iodide-stained nuclei were then analyzed on a BD Accuri C6 flow cytometer (Becton Dickinson) and compared to a diploid that was also co-chopped with the same standard. The fluorescence of each nucleus was quantified (directly proportional to the DNA quantity of the nucleus) and plotted along the x-axis while counts of nuclei were plotted on the y-axis creating a histogram. The values of the mean of a sample/mean of the standard was compared to the mean of a diploid/mean of the standard. Haploids would have a value of half that of the diploid. In FIG. 1A, the sample identified was a diploid because as compared to the diploid control, a 2n peak was observed for this sample. In FIG. 1B, the sample identified was a haploid because as compared to the diploid control, a separate and distinct peak, n, was observed for this sample.

The haploid genotype was further confirmed with a molecular markers analysis.

### Example 3. Production of Double Haploid Sorghum Plants

After identifying haploid plants, these haploids can be doubled to generate a plant that is homozygous at all loci in the nuclear genome. The haploid seedlings can be treated with a doubling agent, such as colchicine or other cell division inhibitors. The use of doubling agents and methods of use to create double haploids are well known in the art.

Briefly, the haploid seedlings are treated with colchicine (or any similar chromosome doubling agent) for 12 hours and then thoroughly washed with distilled water. After the treatment, the seedlings are transferred back to the greenhouse and planted in pots with soil. Three to four weeks later the well-established seedlings could be transplanted to the field.

As an alternative to the treatment of seeds or seedlings, new double haploid plants can be regenerated out of anthers, pollen or egg cells of haploid plants subjected to heat or cold pre-treatments, or also treated with colchicine or any other antimitotic agent.

Having described the present disclosure and inventions in detail, it will be apparent that modifications, variations, and equivalent embodiments are possible without departing from the spirit and scope of the present disclosure as described herein and in the appended claims. Furthermore, it should be appreciated that all examples in the present disclosure are provided as non-limiting examples.

What is claimed is:

1. A mutated polynucleotide encoding a mutated patatin phospholipase protein wherein the said protein comprises aspartic acid (D) at position 41 instead of glycine (G).

2. The polynucleotide as claimed in claim 1, comprising a nucleotide sequence according to SEQ ID NO: 1 or 11, or a nucleotide sequence which is at least 80% identical to SEQ ID No.: 1 or 11.

3. The polynucleotide as claimed in claim 1 encoding the amino acid sequence according to SEQ ID NO: 2, or an amino acid sequence being at least 90% identical to SEQ ID NO:2.

4. A mutated patatin phospholipase protein, comprising an amino acid substitution from glycine (G) to aspartic acid (D) at position 41.

5. The mutated patatin phospholipase protein as claimed in claim 4, comprising the amino acid sequence according to SEQ ID NO: 2.

6. A sorghum haploid inducer line comprising one or more mutations in the endogenous gene encoding a patatin phospholipase,
wherein the one or more mutations lead to at least the amino acid substitution from glycine (G) to aspartic acid (D) at position 41 of the papatin phospholipase protein.

7. The sorghum haploid inducer line as claimed in claim 6, wherein the patatin phospholipase protein is encoded by the nucleotide sequence of SEQ ID NO:1 or 11 or comprises the amino acid sequence according to SEQ ID NO:2.

8. The sorghum haploid inducer line of claim 6, wherein the haploid induction rate is greater than 2%.

9. The sorghum haploid inducer line of claim 6, wherein the haploid induction rate is greater than 5%.

10. The sorghum haploid inducer line of claim 6, wherein the haploid induction rate is between 2% and 10%.

11. A sorghum plant comprising the mutated polynucleotide as claimed in claim 1.

12. The sorghum plant of claim 11, which, when crossed with a wildtype diploid plant, generates haploid progeny.

13. A method of generating an F1 progeny plant having half the ploidy of a parent plant expressing an endogenous wildtype patatin phospholipase protein, the method comprising,
a) crossing the parent plant to a plant of the haploid inducer line as claimed in claim 6; and
b) selecting F1 progeny generated from the crossing step having half the ploidy of the parent plant.

14. The method of claim 13, wherein the parent plant is the ovule parent.

15. The method of claim 14, which further comprises converting at least one selected haploid plant into a doubled haploid plant.

16. The method of claim 13, wherein the wildtype patatin phospholipase protein comprises SEQ ID NO: 10.

17. A method of detecting the polynucleotide as claimed in claim 2 comprising the use of primers according to SEQ ID NO: 4, 5, 6 and combinations thereof.

18. A method of selecting the sorghum plant as claimed in claim 11, which comprises using flow cytometry, phenotypic selection or combinations thereof.
